(19) United States
(12) Patent Application Publication
(10) Pub. No.: US 2009/0299488 A1
(43) Pub. Date: Dec. 3, 2009

(54) PROSTATIC STENT

(76) Inventor: Marian Devonec, Miribel (FR)

Correspondence Address: CANTOR COLBURN, LLP, 20 Church Street, 22nd Floor, Hartford, CT, 06103, US

(21) Appl. No.: 12/302,084

(22) PCT Filed: May 22, 2007
(86) PCT No.: PCT/IB07/02172

§ 371 Date: Jul. 1, 2009

Related U.S. Application Data
(60) Provisional application No. 60/802,112, filed on May 22, 2006.

(51) Int. Cl. A61F 2/04

(52) U.S. Cl. 623/23.66

(57) ABSTRACT

The invention concerns a prostatic stent including a flexible tubular member having a lateral wall equipped with anchoring means, adapted for anchoring the prostatic stent into the urethra, wherein the anchoring means includes a succession of hollow and projecting elements defining a continuous and smooth anchoring profile, thereby forming an anchor for the prostatic stent in place into the urethra, and adapted to avoid any lesion of the urethra during insertion or withdrawal of the stent.

### TECHNICAL FIELD OF THE INVENTION

The invention concerns a prostatic stent, in particular a prostatic stent able to be used in combination with an introducer in order to treat bladder outlet obstruction, as generally described in WO 2006126060, the content of which is hereby incorporated by reference.

### BRIEF DISCUSSION OF RELATED ART

A prostatic stent known from the prior art is disclosed in WO 91/00712. Such prostatic stent comprises a flexible tubular member having a lateral wall and the bladder side of the stent equipped with anchoring means, adapted for anchoring the prostatic stent into the urethra.

The stent is able, due to the flexibility of the tubular member, to accommodate the natural anatomical bend of the prostatic urethra.

The anchoring means are twofold:
the first anchoring means is formed by a roughened outer jacket, mounted on the tubular element. The outer jacket comprises a plurality of sharp elements extending outwardly or inwardly, thereby providing anchoring into the urethra.

However, such anchoring means generally cause damage to the urethra during insertion or withdrawal of the stent. Indeed, the mucous membrane can be torn by the sharp anchoring means. In this case, blood clot can appear, thereby obstructing the lumen of the stent and the urethra.
the second anchoring means is a circular flange located on the bladder side of the stent. The flange in contact with the bladder (neck) orifice has an outer diameter larger than the diameter of the bladder orifice and thus prevents the stent from downward migration.

With this concept the length of the stent must be adapted to the exact length of the prostatic urethra. A given prostate will be best stented by a stent with the appropriate length: too short a stent may not release the obstruction, too long it crosses through the urinary sphincter resulting in patient's incontinence.

For this reason a set of stents with lengths increasing by increments of 5 to 10 mm must be available with this concept. The length of the obstructed urethra has to be measured by endoscopy or ultrasonography to choose among the set of stents ranging from 30 up to 80 mm length, the stent with the appropriate length for a given prostate.

### BRIEF SUMMARY OF THE INVENTION

The invention intends to avoid such drawbacks by proposing a prostatic stent offering a sufficient anchoring into the prostatic urethra without damaging it during its insertion or removal and a prostatic stent with a single length allowing the catheterization of the majority of prostates enlarged by benign prostatic hypertrophy.

To this end, the invention concerns a prostatic stent of the above mentioned type, characterized in that said anchoring means comprise a succession of projecting and hollow elements defining a continuous and smooth anchoring profile, thereby forming an anchor for the prostatic stent in place into the urethra, and adapted to avoid any lesion of the urethra during insertion or withdrawal of the stent.

After insertion of the stent, the mucous membrane tends to conform the wavy profile of the stent so that the membrane penetrates the hollow elements and that the projecting elements cooperate with the membrane to enhance the anchoring.

Preferably, the anchoring means comprise an helical groove.

Such helical groove allows the stent to be sufficiently flexible and also to be rigid enough in order to be easily inserted into the urethra. Such groove also provides a low pressure contact with the lumen minimizing prostatic urethra irritation. The groove also facilitates an angulation of the tubular member by more than 90 degrees without closure of the lumen of the tubular member.

In another embodiment, the anchoring means comprise an accordion pleated groove.

In this embodiment, the stent can eventually keep its bent shape once shaped. This contributes to better anchoring inside the prostatic urethra. The stent returns to the straight state during retrieval of the stent.

Preferably, the tubular member comprises a distal end closed by a closing member, the lateral wall comprising at least one aperture enabling the urine from the bladder to flow through the tubular member.

The closed distal end provides an abutment for the distal end of a stylet. The use of a stylet is well know from the prior art.

According to one embodiment of the invention, the tubular member and the closing member are made out of one piece.

According to another embodiment of the invention, the tubular member and the closing member are two distinct parts, the closing member being fixedly engaged on the tubular member, notably by welding or by over moulding.

Preferably, the tubular member comprises an opened distal end.

According to another embodiment of the invention, the proximal end of the tubular member is equipped with flexible tongues, each of said tongues being arranged to be elastically biased toward a radial and outward position.

The flexible tongues are forming an half-Malecot part which is able to be deployed at the level of the veru montanum in the prostatic portion of the urethra. The tongues, due to their specific orientation and their flexibility, tend to flatten during insertion of the stent whereas they spread at the level of the veru montanum as soon as the stent has been released. In the released state, the tongues prevent the stent from downward migration. Mild traction on the thread attached to the proximal end of the stent results in the abutment of the tongues above the striated sphincter, which means correct positioning.

According to one particular embodiment of the invention, the lateral wall is equipped with at least one multi-winged Malecot.

The Malecot also prevent the stent from downward migration. In this case, mild traction on the thread attached to the proximal end of the stent results also in the flattening of the wings of the Malecot. Thus abutment against the sphincter is not perceptible.

Preferably, the length of the stent is in the range of 80 mm.

Such length allows catheterization of the prostatic urethra of the majority of patients using this single or unique dimension. Indeed, the dimensions of the prostate may differ from one patient to another. It is important that the distal end of the stent is located into the bladder. However, the length of the part of the stent which projects from the prostatic urethra into the bladder does not have to be taken into account by the physician because any patient will easily tolerate a 10 to 50 mm projecting part of the stent inside the bladder.

Fifty millimetres is the length which will protrude inside the bladder of a patient with a normal size prostate, but with a bladder orifice sclerosis. A 50 mm length is the common length of Foley catheter protruding inside the bladder (diameter of the inflated balloon plus tip of the Foley catheter).

The physician is therefore not obliged to use a set of stents of different lengths and to measure the length of the prostatic urethra in order to determine which stent could be the most adapted to the patient's prostate.

Preferably, at least the tubular member is made of silicone.

In another embodiment of the invention, the length of the stent is in the range of 80 mm.

Preferably, the tubular element comprises a portion able to elongate along its longitudinal axis.

It is therefore possible to adjust the length of the stent with regard to the size of the biggest prostates.

### BRIEF DESCRIPTION OF THE DRAWINGS

The present invention is now described with reference to annexed drawing wherein:

FIG. 1a is a front elevation of a first embodiment of the stent according to the invention, the stent comprising an helical groove;

FIG. 1b shows a second embodiment in which the stent comprises accordion pleated grooves;

FIG. 2a is a view of the stent in a bent position;

FIG. 2b is a partly cross-section view of the stent in a bent position;

FIG. 3 represents the stent linked to an additional tubular element.

FIGS. 4 to 8 represent other embodiments of the invention.

### DETAILED DESCRIPTION OF THE INVENTION

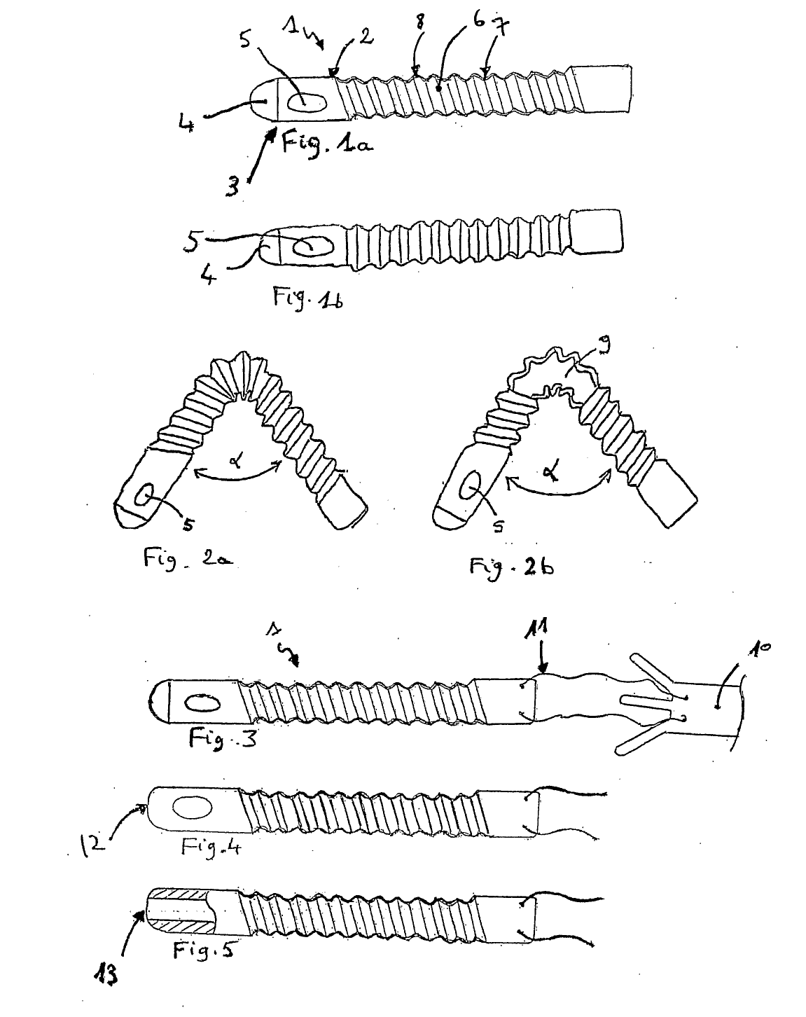

As shown in FIG. 1, the invention concerns a prostatic stent comprising a tubular member 1 made of silicone and having a lateral wall 2.

The inner diameter of the tubular member allows the insertion of a stylet (as shown and described in WO 2006126060) whereas the outer diameter allows the catheterization of most adult patient with a normal urethra, without urethral stricture. It must be understood that the insertion of such a stent can be managed by the way of most of the well known methods, for example as described and claimed in WO 2006126060.

The length of the stent is in the range of 80 mm.

The tubular member 1 comprises a distal part 3 which is cylindrical, the distal end being closed by a closing member 4.

The closing member 4 is fixedly engaged on the tubular member, notably by welding or by over moulding.

The lateral wall 2 comprises two opposite apertures 5 enabling the urine from the bladder to be drained through the tubular member 1.

The tubular member further comprises a median part 6 which is equipped with anchoring means, adapted for anchoring the prostatic stent into the obstructed prostatic urethra.

Said anchoring means comprise an helical grove 7 (FIG. 1a) arranged in form of a screw thread or accordion pleated groves (FIG. 1b) with rounded crests 8, defining a continuous and smooth anchoring with a sinusoidal profile.

Such smooth anchoring profile forms an anchor for the prostatic stent when the stent is in place into the urethra, and is also adapted to avoid any lesion of the urethra during insertion or withdrawal of the stent.

FIG. 2 represents the stent in a bended state, where the tubular member 1 is bent with an angle α less than 90 degrees (FIG. 2a). As it can be seen from FIG. 2b, the lumen 9 formed by the tubular part in the bent region is not closed due to the flexibly of the tubular member 1. In addition, due to the elasticity of the tubular member 1, the latter recovers its original form after bending. This facilitates retrieval of the stent.

As detailed on FIG. 3, the tubular member 1 is able to be linked to an additional tubular member 10 in a manner similar to the known prior art (see for example WO 2006126060) through flexible connecting means 11.

The additional tubular member 10 comprises flanges forming an half Malecot, able to abut against the urinary striated sphincter and to provide a clear tactile feed back to the physician, thereby allowing correct positioning of the stent only by hand without the need of any external equipment to control the positioning.

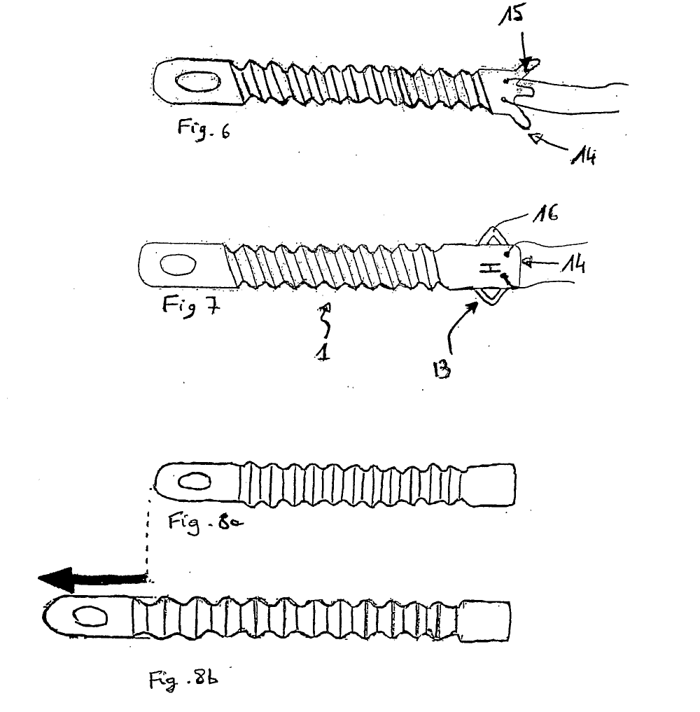

In another embodiment of the invention detailed on FIG. 4, the tubular member 1 and the closing member 4 are made out of one piece. The tubular element then comprises a blind tip 12 of the Foley type.

In a still other embodiment detailed on FIG. 5, the tubular member 1 comprises an opened distal end 13. In such case, there is no need to provide additional apertures in the lateral wall 2.

In another embodiment detailed on FIG. 6, the proximal end 14 of the tubular member is equipped with flexible tongues 15, each of said tongues being arranged to be elastically biased toward a radial and outward position, thereby forming an half Malecot.

In a still other embodiment detailed on FIG. 7, the lateral wall 2 is equipped with a multi-winged Malecot 16. The multi-winged Malecot 16 is located on the proximal end 14 of the tubular element 1.

In another embodiment detailed on FIG. 8, the accordion pleated wall (FIG. 8a) can be stretched longitudinally to increase the length of the stent by 10 to 20 mm (FIG. 8b) in the case of stenting a huge size prostate.

It is obvious that the features of the embodiments detailed on FIG. 3 can be combined with the features of the embodiments detailed on FIGS. 4 to 8.

1. A prostatic stent comprising
a flexible tubular member having a lateral wall equipped with anchoring means, adapted for anchoring the prostatic stent into the urethra,
wherein said anchoring means comprise a succession of hollow and projecting elements defining a continuous and smooth anchoring profile, thereby forming an anchor for the prostatic stent in place into the urethra, and adapted to avoid any lesion of the urethra during insertion or withdrawal of the stent.

2. A prostatic stent according to claim 1, wherein the anchoring means comprise an helical groove.

3. A prostatic stent according to claim 1, wherein the anchoring means comprise an accordion pleated groove.

4. A prostatic stent according to claim 1, wherein the tubular member comprises a distal end closed by a closing member, the lateral wall comprising at least one aperture enabling urine from the bladder to flow through the tubular member.

5. A prostatic stent according to claim 4, wherein the tubular member and the closing member are made out of one piece.

6. A prostatic stent according to claim 4, wherein the tubular member and the closing member are two distinct parts, the closing member being fixedly engaged on the tubular member, by welding or by over moulding.

7. A prostatic stent according to claim 1, wherein the tubular member comprises an opened distal end.

8. A prostatic stent according to claim 1, wherein the proximal end of the tubular member is equipped with flexible tongues, each of said tongue being arranged to be elastically biased toward a radial and outward position.

9. A prostatic stent according to claim 1, wherein the lateral wall is equipped with at least one multi-winged Malecot.

10. A prostatic stent according to claim 1, wherein at least the tubular member is made of silicone.

11. A prostatic stent according to claim 1, wherein a length of the stent is in the range of 80 mm.

12. A prostatic stent according to claim 1, wherein the tubular element comprises a portion able to elongate along a longitudinal axis.
